(19) United States
(12) Patent Application Publication
(10) Pub. No.: US 2025/0032736 A1
(43) Pub. Date: Jan. 30, 2025

(54) NASOPHARYNGEAL AIRWAY

(71) Applicant: TIEN-SHENG CHEN, Taipei City (TW)

(72) Inventor: TIEN-SHENG CHEN, Taipei City (TW)

(21) Appl. No.: 18/767,498

(22) Filed: Jul. 9, 2024

(30) Foreign Application Priority Data
Jul. 25, 2023 (TW) 112127810

(51) Int. Cl. A61M 16/04

(52) U.S. Cl. CPC A61M 16/0461; A61M 16/0475

(57) ABSTRACT

A nasopharyngeal airway has multiple fenestrations and a curved tube. The curved tube has a curved front section with a front end. Multiple fenestrations are provided in the curved front section. The curved tube has a curved tube length DI and the curved front section has a front length D2, where

$$D 2 \leq \frac{1}{3} D 1 .$$

### BACKGROUND OF THE INVENTION

### Field of the Invention

The present invention relates to a nasopharyngeal airway; more particularly, the present invention relates to a nasopharyngeal airway having multiple fenestrations provided in a curved front section.

### Description of the Related Art

A nasopharyngeal airway can help establish a smooth breathing passage from a patient's nostril to the tongue base or laryngopharynx. When a patient is dangerously ill or unconscious, the patient's tongue base may slide back to obstruct the tracheal airway. At this time, it is necessary to insert a nasopharyngeal airway to secure a smooth breathing passage for the patient. However, the ventilation hole of a conventional nasopharyngeal airway is provided only in the tip of a tube body of the conventional nasopharyngeal airway, and the nasopharyngeal airway has a fixed length. As a result, after the nasopharyngeal airway is inserted into the patient's nasal cavity, the healthcare provider will usually encounter difficulty in causing the ventilation hole provided in the tip of the nasopharyngeal airway to pass the tongue base and enter the patient's laryngopharynx. Improper positioning of the ventilation hole will result in failure to establish a smooth breathing passage for providing sufficient oxygen to the patient. Therefore, there is a need to provide a nasopharyngeal airway to mitigate and/or obviate the aforementioned problems.

### SUMMARY OF THE INVENTION

It is an object of the present invention to provide a nasopharyngeal airway having multiple fenestrations provided in a curved front section.

To achieve the abovementioned object, the nasopharyngeal airway of the present invention includes a plurality of fenestrations and a curved tube. The curved tube includes a curved front section, and the curved front section has a front end. The plurality of fenestrations are provided in the curved front section. The curved tube has a curved tube length D1 and the curved front section has a front length D2, where D2≤ ⅓ D1.

According to the characteristics of the present invention, by means of providing a plurality of fenestrations in the curved front section of the nasopharyngeal airway, when the nasopharyngeal airway is placed into a human pharynx and the front end of the nasopharyngeal airway enters a human esophagus, even if the healthcare provider cannot precisely place the ventilation holes of the nasopharyngeal airway of the present invention into the patient's upper airway, the plurality of fenestrations provided in the curved front section can still provide oxygen to the patient, thereby resolving the problem that the conventional nasopharyngeal airway cannot be precisely placed in an oxygen provision position without difficult manipulations.

### BRIEF DESCRIPTION OF THE DRAWINGS

FIG. 1 illustrates a perspective view of a nasopharyngeal airway according to one embodiment of the present invention;

FIG. 2 illustrates another perspective view of the nasopharyngeal airway according to one embodiment of the present invention;

FIG. 3 illustrates a bottom view of the nasopharyngeal airway according to one embodiment of the present invention;

FIG. 4 illustrates a cross-sectional drawing of an curved front section of the nasopharyngeal airway according to the present invention; and

FIG. 5 illustrates a cross-sectional schematic drawing of the nasopharyngeal airway being placed into a human pharynx.

### DETAILED DESCRIPTION OF THE EMBODIMENT

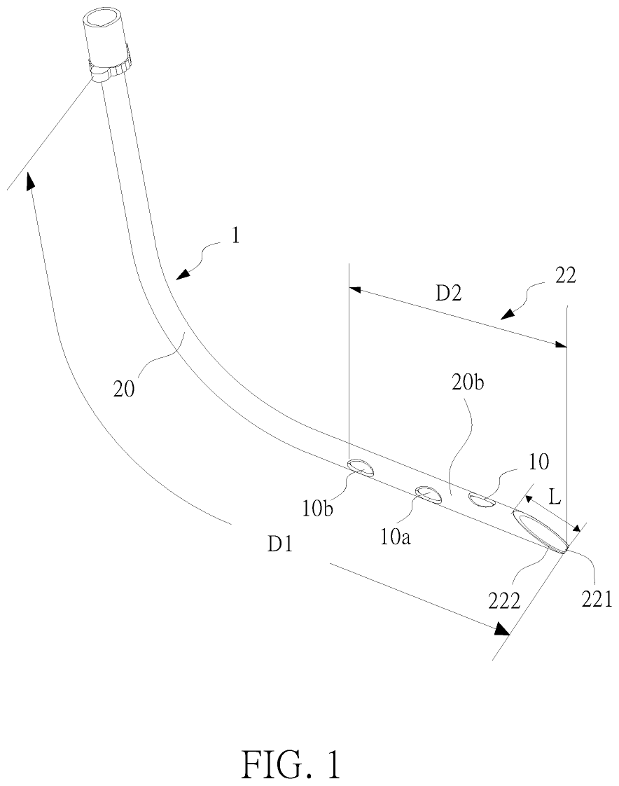

In order to better understand the technical content of the present invention, preferred embodiments are described as follows. Please refer to FIG. 1 to FIG. 5, which illustrate perspective views, a bottom view and a cross-sectional drawing of a curved front section of a nasopharyngeal airway according to the present invention, as well as a cross-sectional schematic drawing of the nasopharyngeal airway inserted into a human pharynx.

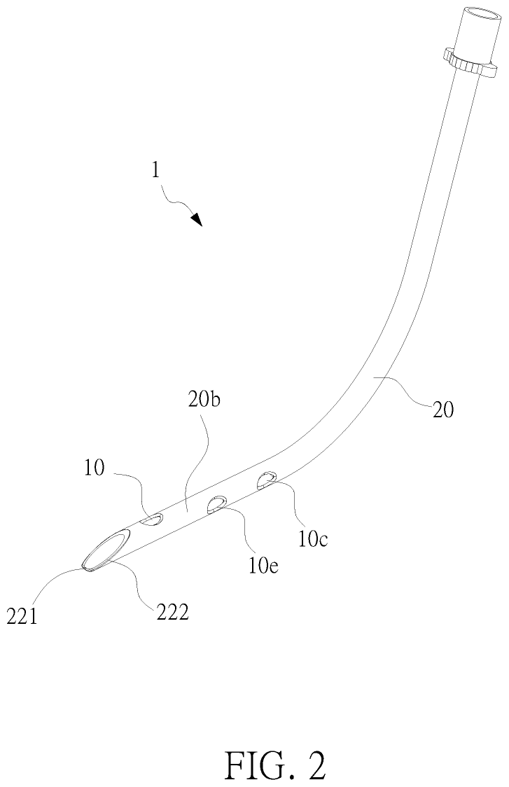

As shown in FIG. 1 and FIG. 2, in this embodiment, the nasopharyngeal airway 1 of the present invention includes a plurality of fenestrations 10, 10a, 10b and a curved tube 20. The curved tube 20 includes a curved front section 22. The curved front section 22 includes a front end 221 and an oblique surface 222. The fenestrations 10, 10a, 10b are provided in the curved front section 22. The curved tube 22 has a curved tube length D1 and the curved front section 22 has a front length D2, where

$$D 2 \leq \frac{1}{3} D 1 .$$

According to one embodiment of the present invention, the curved tube length D1 can be 12 cm<D1<18 cm or 14 cm<D1<18 cm; the front length D2 can be 2 cm <D2<6 cm or 3 cm<D2<6 cm. However, the curved tube length D1 and the front length D2 are not limited to the abovementioned length ranges, for the curved tube length D1 and the front length D2 may vary as long as the criterion of

$$D 2 \leq \frac{1}{3} D 1$$

is satisfied. As shown in FIG. 1, the front length D2 is the distance between the front end 221 and the farthest opening 10b. In this embodiment, the length L of the oblique surface 222 is between 0.6 cm and 2.5 cm, or between 1 cm and 2.5 cm, and the opening 10 closest to the front end 221 is disposed at a distance of 1 cm to 1.5 cm from the front end 221. In other words, the nasopharyngeal airway 1 of the present invention provides an opening 10 at a position 1 cm to 1.5 cm from the front end 221. Please note the arrangements of the fenestrations are not limited to the above embodiment, for the opening 10 can also be provided directly at the front end 221, and the fenestrations can be provided within the front length D2.

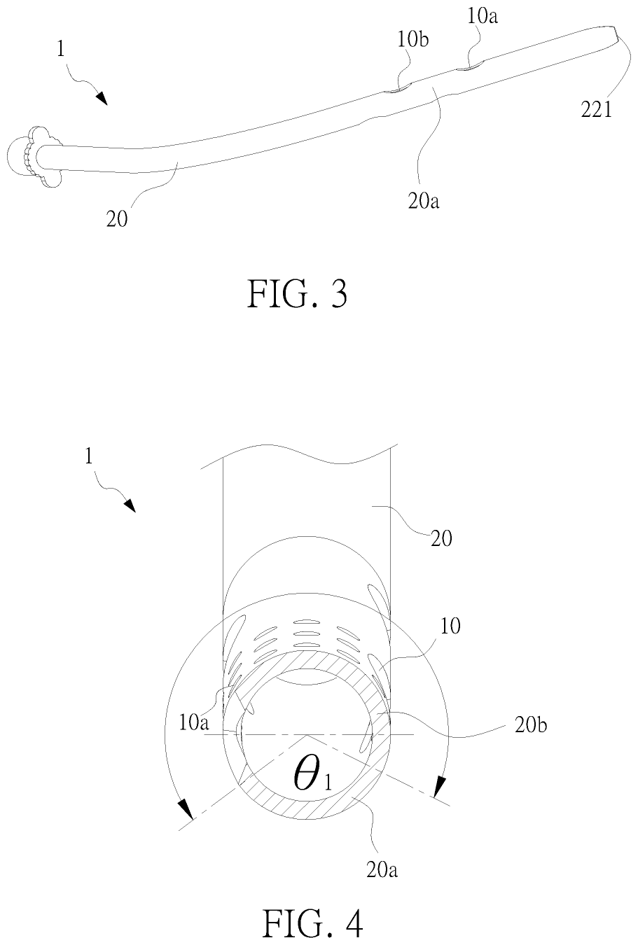

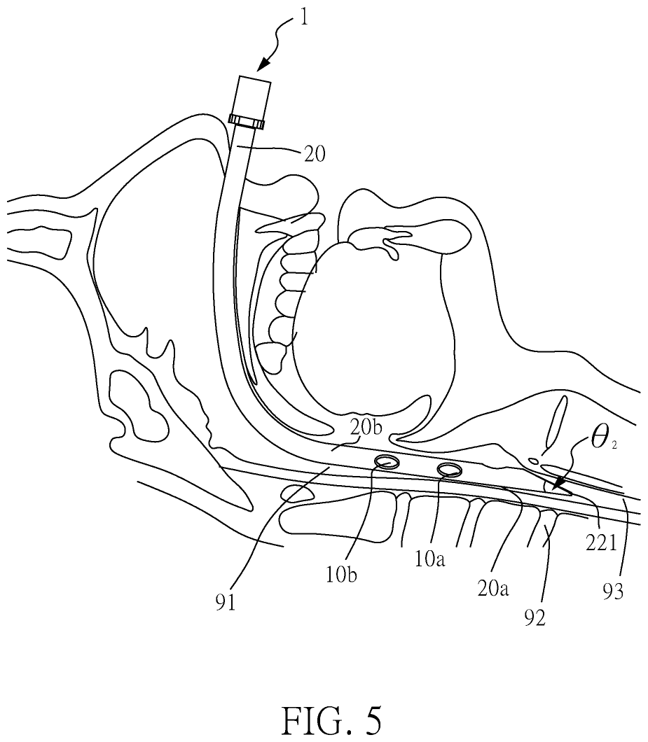

As shown in FIG. 3 and FIG. 4, the curved tube 20 has a curved inner side 20a and a curved outer side 20b. No fenestrations are provided in the curved inner side 20a. The fenestrations 10, 10a, 10b, 10c are provided discretely in the curved outer side 20b at distances of 1 cm to 1.5 cm from the front end 221. As shown in FIG. 5, after the nasopharyngeal airway 1 of the present invention is placed into a human pharynx 91 via the nasal cavity, the curved outer side 20b is the side of the nasopharyngeal airway 1 adjacent to the human pharynx 91, and the curved inner side 20a is the side of the nasopharyngeal airway 1 adjacent to the human cervical vertebra 92. When the nasopharyngeal airway 1 is placed into the human pharynx 91, in this embodiment, the length of the front end 221 of the nasopharyngeal airway 1 of the present invention can enter the human esophagus 93 such that the fenestrations 10a, 10b located in the curved outer side 20b can provide oxygen for the patient in the upper airway. In order to secure the ventilation volume of the nasopharyngeal airway 1 of the present invention, an open area of the plurality of fenestrations 10, 10a, 10b, 10c is equal to 3% to 40%, 10% to 40% or 10% to 30% of the area of the curved front section 22. Alternatively, it is also feasible to provide at least one and at most five fenestrations discretely within the unit area of one square centimeter of the curved front section 22. Please note that, according to one embodiment of the present invention, the curved inner side 20a and the curved outer side 20b can be observed in the cross-sectional drawing of the curved tube 20 as shown in FIG. 4. If the cross-sectional axle center is designated as the circle center, the curved inner side 20a covers the arc-shaped area within the central angle θ₁, which is around 120 degrees, under the circle center, as shown in FIG. 4, and the curved outer side 20b covers all of the remaining area by excluding the area covered by the curved inner side 20a. In FIG. 5, the inclination angle θ₂ of the oblique surface 222 is, but is not limited to, around 45 degrees. The inclination angle θ₂ is between 80 degrees and 20 degrees.

Because of the provision of fenestrations in the curved front section 22 of the nasopharyngeal airway 1 of the present invention, as well as the insertion of the front end 221 into the human esophagus 93 when the nasopharyngeal airway 1 is placed into the human pharynx 91, even if the healthcare provider cannot precisely position the ventilation hole located in the front tip of the nasopharyngeal airway 1 of the present invention in the patient's upper airway, oxygen can still be provided to the patient via the opening 10 provided in the curved front section 22. As a result, the risk of oxygen deprivation due to inaccurate nasopharyngeal airway positioning is reduced, and the lack of precision in placement of the nasopharyngeal airway in an oxygen provision position is therefore resolved.

Although the present invention has been explained in relation to its preferred embodiments, it is to be understood that many other possible modifications and variations can be made without departing from the spirit and scope of the invention as hereinafter claimed.

What is claimed is:

1. A nasopharyngeal airway, comprising:
a plurality of fenestrations; and
a curved tube, the curved tube including an curved front section and the curved front section including a front end, wherein the plurality of fenestrations are provided in the curved front section, the curved tube has a curved tube length D1, and the curved front section has an front length D2, where
$$D 2 \leq \frac{1}{3} D 1 .$$

2. The nasopharyngeal airway as claimed in claim 1, wherein an open area of the plurality of fenestrations covers 3% to 40% of the area of the curved front section.

3. The nasopharyngeal airway as claimed in claim 2, wherein when the nasopharyngeal airway is placed into a human pharynx, the front end enters a human esophagus.

4. The nasopharyngeal airway as claimed in claim 2, wherein the curved tube length D1 is 10 cm<D1<22 cm.

5. The nasopharyngeal airway as claimed in claim 4, wherein when the nasopharyngeal airway is placed into a human pharynx, the front end enters a human esophagus.

6. The nasopharyngeal airway as claimed in claim 4, wherein a plurality of fenestrations are provided near the front end.

7. The nasopharyngeal airway as claimed in claim 6, wherein when the nasopharyngeal airway is placed into a human pharynx, the front end enters a human esophagus.

8. The nasopharyngeal airway as claimed in claim 6, wherein the curved tube has a curved inner side and a curved outer side, and the plurality of fenestrations are provided in the curved outer side.

9. The nasopharyngeal airway as claimed in claim 8, wherein when the nasopharyngeal airway is placed into a human pharynx, the front end enters a human esophagus.

10. The nasopharyngeal airway as claimed in claim 8, wherein each of the fenestrations is provided discretely in the curved outer side at a distance of 1 cm to 1.5 cm from the front end.

11. The nasopharyngeal airway as claimed in claim 10, wherein when the nasopharyngeal airway is placed into a human pharynx, the front end enters a human esophagus.

12. The nasopharyngeal airway as claimed in claim 1, wherein within the unit area of one square centimeter of the curved front section, at least one and at most five fenestrations are provided discretely therein.

13. The nasopharyngeal airway as claimed in claim 1, wherein the curved front section comprises an oblique surface, and an inclination angle θ₂ is between 80 degrees and 20 degrees.

14. The nasopharyngeal airway as claimed in claim 13, wherein the inclination angle θ₂ is 45 degrees.

15. The nasopharyngeal airway as claimed in claim 1, wherein when the nasopharyngeal airway is placed into a human pharynx, the front end enters a human esophagus.
